(19) United States
(12) Patent Application Publication
(10) Pub. No.: US 2020/0261354 A1
(43) Pub. Date: Aug. 20, 2020

(54) COMPOSITIONS FOR TREATING PERIODONTAL DISEASES

(71) Applicant: GR Biosysystems, Inc., South Jordan, UT (US)

(72) Inventors: Andrew J. GROSS, South Jordan, UT (US); Catalina MATIAS, South Jordan, UT (US); Shane RIRIE, South Jordan, UT (US)

(21) Appl. No.: 16/496,559

(22) PCT Filed: Mar. 21, 2018
(86) PCT No.: PCT/US2018/023469

§ 371 Date: Sep. 23, 2019

Related U.S. Application Data
(60) Provisional application No. 62/474,456, filed on Mar. 21, 2017.

(51) Int. Cl. A61K 9/00; A61K 31/12; A61K 31/7004; A61K 31/16; A61K 36/9066; A61K 47/59; A61K 9/06

(52) U.S. Cl. CPC A61K 9/0014; A61K 31/12; A61K 31/7004; A61K 9/06; A61K 36/9066; A61K 47/593; A61K 9/0053; A61K 31/16

(57) ABSTRACT

The present disclosure generally relates to compositions that inhibit matrix metalloproteinases (MMPs). These compositions may be particularly useful in treating periodontal disease and skin wrinkling. The compositions can be prepared as a topical formulation, ointment, mouthwash, or packaged in a syringe.

### REFERENCE TO EARLIER FILED APPLICATION

This application claims the benefit under 35 U.S.C. § 119(e) of U.S. Provisional Application No. 62/474,456, filed Mar. 21, 2017, the disclosure of which is incorporated, in its entirety, by this reference.

### BACKGROUND

### 1. Field of the Invention

The present disclosure generally relates to compositions for inhibiting matrix metalloproteinases (MMPs). More particularly the disclosure relates to compositions including an MMP inhibitor for treating periodontal diseases.

### 2. Description of the Related Art

Periodontitis is a chronic inflammatory disease characterized by the destruction of the periodontium due to an excessive and sustained host response to a multi-microbial insult. It affects around 64.7 million adults in the United States, and it is the lead cause of edentulism in the developed world.

The predominant paradigm for the etiology of periodontitis is the presence of a biofilm composed by what is known as the red complex. A combination of microbes including Porphyromonas gingivalis, Treponema denticola, and Tanerella forsythia. P. gingivalis was a widely accepted model for periodontal inflammation, as it is easily cultured and causes inflammatory bone loss. Currently, the polymicrobial synergy and dysbiosis model (PSD) is the mainstream mechanism in the etiology of periodontitis. The PSD model compares the combination of several bacterial species in periodontal disease with their relative abundance in oral health. New sequencing techniques permitted the identification of diverse microbial communities involved in periodontitis. In a susceptible host, keystone pathogens such as P. gingivalis initiate a breakdown in homeostasis while former commensals become proinflammatory pathobionts that cause a dysbiotic state that promotes periodontal disease.

While bacteria is essential for periodontitis to occur, the severity, pattern, and progression of the disease is not solely determined by the microbial burden alone, but also an overwhelming host inflammatory response. The host response can vary even in two individuals with similar periodontopathogenic profiles. Initially, a pathogen such as P. gingivalis interacts with Toll-like receptors 2 and 4 (TLR2 and TLR4) from local cells, exploiting the TLR2/TLR4 crosstalk with the complement system (C5a) to hijack normal defense responses and chemotaxis of defense cells. Meanwhile, other virulence factors induce the production of inflammatory cytokines (interleukins, tumor necrosis factor-α), prostanoids and proteolytic enzymes, mainly matrix metalloproteinases (MMPs) that are the main culprits of gingival damage.

MMPs are calcium-dependent zinc-containing endopeptidases that degrade extracellular matrix (ECM), initially discovered by Gross and Lapiere in 1962. Additionally, metalloproteinase activity is now linked to the control of immune responses. Post-translational modification of proteins and activation of signal transduction pathways that control cytokine biosynthesis allow the MMPs to direct systemic inflammation or barrier immunity. As it is evident from their substrates, MMPs have a vast proteolytic potential that include collagen types I-XVII, pro forms of inflammatory molecules such as tumor necrosis factor (TNF), interleukin 1β (IL-1β), monocyte chemoattractant protein (MCP) and even other pro forms of MMPs. When present in excess, MMPs severely compromise tissue function and integrity.

Most MMPs present with four distinct functional domains: signal peptide, propeptide, catalytic domain and hemopexin-like domain. All of the MMPs contain a highly homologous catalytic domain and a propeptide. The propeptide interacts with the Zn²⁺ ion located in the catalytic pocket through a cysteine residue and keeps the enzyme in a latent, inactive state. The propeptide must be removed to allow for enzyme activity through a pathway that varies according to the MMP subfamily.

Periodontal diseases may also be influenced by the presence of iron. Iron is an essential trace element involved in a wide range of biological processes, such as oxygen transport, energy production and host defense. Paradoxically, iron is also a potent oxidative agent capable of catalyzing the production of reactive oxygen species (ROS) and thus also contributes to cytotoxicity and tissue damage.

Bacteria require iron from their mammalian hosts in order to replicate and survive. In mammals, however, iron is not easily accessible due to highly regulated mechanisms that seek to make iron unavailable to invading pathogens. In chronic periodontitis the iron concentration in the gingival crevicular fluid (GCF) is significantly elevated compared to that in the plasma, promoting bacterial growth.

The current status of periodontitis treatment is based in mechanical debridement of biofilm (scaling and root planning), systemic or localized antibiotic therapy and even antimicrobial photodynamic therapy. Surgical procedures such as gingivectomy and flap debridement are used with less frequency and often accompanied by antimicrobial therapy. The sole focus of these approaches is to control the microbial invasion or repair tissue and they do not address the feedback from the host response that perpetuates the disease. Although both non-surgical and surgical approaches can be effective in controlling periodontal damage, they require strict maintenance regimes and do not prevent disease in other sites.

As a response to the limitations of the traditional therapies, new agents have been used in preclinical and clinical studies, namely host-modulatory agents, including anti-proteinase agents, anti-inflammatory agents and anti-resorptive agents. New therapeutics approaches are needed that focus on changing the inflammatory process, as opposed to focusing solely on the microbial insult. Effective control of the immune response may slow down the disease progression, improve clinical outcomes and even prevent future sites of active periodontitis.

### BRIEF SUMMARY

In some embodiments, a topical, pharmaceutical composition is disclosed that includes a pharmaceutically acceptable carrier and a compound selected from 1,2,3,4,6-penta-O-galloyl-β-D-glucose, curcumin, deferoxamine, chloromethyl ketone, or any combination thereof.

In some embodiments, a method of treating periodontal disease is disclosed that includes administering to a subject the composition described above.

In some embodiments, a method of treating a wound, fistula, or ulcer is provided that includes administering to a subject a topical composition described above.

The foregoing has outlined rather broadly the features and technical advantages of the present disclosure in order that the detailed description that follows may be better understood. Additional features and advantages of the disclosure will be described hereinafter that form the subject of the claims of this application. It should be appreciated by those skilled in the art that the conception and the specific embodiments disclosed may be readily utilized as a basis for modifying or designing other embodiments for carrying out the same purposes of the present disclosure. It should also be realized by those skilled in the art that such equivalent embodiments do not depart from the spirit and scope of the disclosure as set forth in the appended claims.

### BRIEF DESCRIPTION OF THE DRAWINGS

A detailed description of the invention is hereafter described with specific reference being made to the drawings in which:

FIG. 1 shows the effects of poly galloyl glucopyranose (PGG) on MMP-2 expression;

FIG. 2 shows the effects of PGG on MMP-13 protein expression;

FIG. 3 shows the effects of PGG on MMP-14 protein expression;

FIG. 4 shows the effects of PGG on MMP-3 protein expression;

FIG. 5 shows the effects of PGG on MMP-9 expression;

FIG. 6 shows the effects of PGG on MMP gene expression;

FIG. 7 shows the effects of chloromethyl ketone (CMK) and deferoxamine (DES) on MMP-2 protein expression;

FIG. 8 shows the effects of CMK and DES on MMP-13 protein expression;

FIG. 9 shows the effects of CMK and DES on MMP-14 protein expression;

FIG. 10 shows the effects of CMK and DES on MMP-9 protein expression;

FIG. 11 shows the effects of CMK and DES on MMP-3 protein expression;

FIG. 12 shows the effects of DES on MMP-2 protein expression;

FIG. 13 shows the effects of DES on MMP-9 protein expression;

FIG. 14 shows the effects of DES on MMP-14 protein expression;

FIG. 15 shows the effects of curcumin (Cur) on MMP-2 protein expression;

FIG. 16 shows the effects of Cur on MMP-3 protein expression; and

FIG. 17 shows the effects of CMK and DES on MMP gene expression.

FIG. 18 shows a prior art depiction of a syringe; and

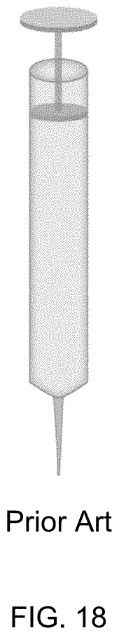

FIG. 19 shows a prior art depiction of a patch.

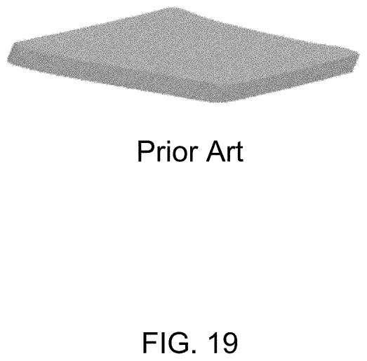

### DETAILED DESCRIPTION

Various embodiments are described below with reference to the drawings. The relationship and functioning of the various elements of the embodiments may better be understood by reference to the following detailed description. However, embodiments are not limited to those illustrated in the drawings. It should be understood that the drawings are not necessarily to scale, and in certain instances details may have been omitted that are not necessary for an understanding of embodiments disclosed herein, such as—for example—conventional fabrication and assembly.

Periodontal diseases affect millions of people and can lead to endentulism. Affected persons usually receive antibiotics or an attempt is made to remove bacteria from affected areas in the oral cavity. Current treatments, however, fail to directly influence the host immune response to the invasive bacteria. The present disclosure relates to compositions that can inhibit MMPs and reduce collagen destruction. The compositions may include poly galloyl glucopyranose, curcumin, chloromethyl ketone, deferoxamine, and combinations of the same.

Poly galloyl glucopyranose or 1,2,3,4,6-penta-O-galloyl-β-D-glucose (PGG, formula (I) shown below) is a polyphenolic gallotannin synthesized by plants. It was initially extracted from Rhus typhina (sumac) in 1990 by Hofmann and Gross.

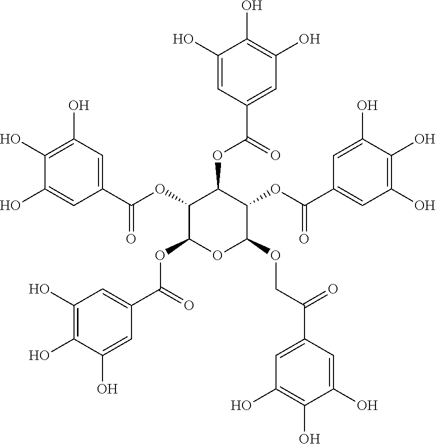 [SMILES: O=C(CO[C@H]1O[C@@H](OC(=O)c2cc(O)c(O)c(O)c2)[C@H](OC(=O)c2cc(O)c(O)c(O)c2)[C@@H](OC(=O)c2cc(O)c(O)c(O)c2)[C@@H]1OC(=O)c1cc(O)c(O)c(O)c1)c1cc(O)c(O)c(O)c1]

PGG has been regarded as an inflammatory promoter for treatment of peripheral blood mononuclear cells (PBMCs) with PGG resulted in the production of TNF-α and IL-β.

Posterior in vitro and in vivo studies in PBMCs demonstrated that PGG could attenuate the inflammatory effect of lipopolysaccharide (LPS), the major component of the cell wall of Gram-negative bacteria. Studies have shown that PGG largely suppressed LPS-induced TNF-α production by as much as 90% with doses as low as 5 μM. In peritoneal and colonic macrophages, PGG did not interfere with the binding of LPS to the toll-like receptors 4 (TLR4) but interacted directly with MyD88 adaptor protein thereby decreasing the production of TNF-α, IL-1β and IL-6.

In the present disclosure, we disclose that PGG and other compounds act as an inhibitor for the protein and gene expression of MMPs, and MMP expression is clinically relevant to the progression of periodontitis. For the purposes of this disclosure PGG may also refer to variants of the structure depicted above, for example variants or PGG-like molecules may include structures where the some or all the outer hydroxyl groups are replaced with a C₁-C₈ alkyl group, where the alkyl group may be methyl. Other PGG-like molecules may have several hydroxyl groups removed from the outer phenyl rings.

In some embodiments, a topical composition is disclosed that includes a pharmaceutically acceptable carrier and a compound selected from PGG, curcumin, deferoxamine, chloromethyl ketone, and combinations thereof.

In some embodiments, the compound is PGG. In some embodiments, the compound is curcumin. In some embodiments, the compound is deferoxamine. In some embodiments, the compound is chloromethyl ketone.

In some embodiments, the composition includes a mixture of two or more of PGG, deferoxamine, chloromethyl ketone, and curcumin. In some embodiments, the mixture is PGG and deferoxamine. In some embodiments, the mixture is PGG and chloromethyl ketone. In some embodiments, the mixture is PGG and curcumin. In some embodiments, the mixture is deferoxamine and chloromethyl ketone. In some embodiments, the mixture is deferoxamine and curcumin. In some embodiments, the mixture is chloromethyl ketone and curcumin.

In some embodiments, the mixture is PGG, deferoxamine, and chloromethyl ketone. In some embodiments, the mixture is PGG, deferoxamine, and curcumin. In some embodiments, the mixture is PGG, chloromethyl ketone, and curcumin. In some embodiments, the mixture is deferoxamine, chloromethyl ketone, and curcumin. In some embodiments, the mixture is PGG, deferoxamine, chloromethyl ketone, and curcumin.

In some embodiments, the composition may include from 0.00001% to 50% by volume of the active compound or compounds. The composition may include from 0.00001% to 30% by volume, 0.00001% to 20% by volume, or 0.00001% to 10% by volume of the compound. In some embodiments, the composition may include 0.001% to 30% by volume, 0.01% to 30% by volume, 0.1% to 30% by volume, or 1% to 30% by volume of the active compound or compounds.

In some embodiments, the pharmaceutically acceptable carrier is selected from poly(glycolide-co-dl-lactide) (PGLA), polyethylene glycol, collagen, hyaluronic acid, liposome, micelle, dendrimer, and combinations thereof.

In some embodiments, the pharmaceutically acceptable carrier is PGLA. The PGLA polymer may be in the form of a microsphere. PGLA is a bioerodible polymer that can be prepared by ring-opening copolymerization of glycolide and lactide. Glycolide and lactide exist in two steroisomeric forms: D and L. D,L lactide may be especially useful in producing copolymers for drug delivery applications. PGLA microspheres may be prepared by water/oil/water (W/O/W) and solid/oil/water (S/O/W) double emulsion solvent evaporation method or other methods such as nanoprecipitation, emulsion, solvent diffusion, or salting-out. The compounds of this disclosure may be incorporated into the matrix of a PGLA microsphere or contained within an aqueous core of the microsphere.

In some embodiments, the compounds may be encapsulated within a liposome or micelle. Encapsulation provides extended and controlled release of the compounds at the application site. Methods of preparing liposomes and micelles are commonly known in the art.

In some embodiments, the composition may also include an antibiotic, anti-inflammatory, analgesic agent, or any combination thereof. The anti-inflammatory may be a corticosteroid such as but not limited to hydrocortisone or triamcinolone acetonide. The analgesic agent may be lidocaine, articaine, mepivacaine, bupivacaine, salts thereof, or mixtures thereof. The analgesic agent may be co-administered with epinephrine. The antibiotic may be tetracycline, doxycycline, metronidazole, clindamycin, amoxicillin and clavulinic acid (augmentin), azithromycin, metronidazole, spiramycin, minocycline, or any combination thereof.

In some embodiments, the composition may include water, a buffer, or a flavoring. A buffer may be included to maintain a pH of the composition. The composition may have a pH ranging from about 4 to about 9.

In some embodiments, a method is disclosed for treating periodontal disease. The method includes administering to a subject the any compositions described above. The subject may be a human or other mammal. In some embodiments, the subject is a human. In some embodiments, the subject is a canine. In some embodiments, the subject is a feline. In some embodiments, the subject may be a horse. The compositions may be administered orally or topically.

In some embodiments, the composition may be in the form of a mouthwash. In some embodiments, the composition may be in the form of an ointment, gel, foam, or spray.

In some embodiments, the periodontal disease may be gingivitis, early periodontitis, moderate periodontitis, or advanced periodontitis.

In some embodiments, the composition may be administered sub-gingivally. The composition may contact human gingival fibroblasts or mucosal fibroblasts. In some embodiments, the composition may be loaded into a syringe. The syringe may comprise a needle that can be applied to the region of the oral cavity that is diseased.

In some embodiments, a method is disclosed for treating a wound, fistula, or ulcer. The method may include administering to a subject a topical composition. The composition may be any composition described above.

In some embodiments, the composition may be in the form of a patch. The patch may be applied to wounds on the skin or inside the mouth. The patch may include an adhesive so that once it is applied the patch can remain at the treated site.

In some embodiments, the composition may contact keratinized, parakaratinized, non-keratinized epithelium and combinations of the same.

In certain embodiments, a method is provided for preventing or treating wrinkles. The method may include applying to skin any composition described in this application. The compounds PGG, curcumin, deferoxamine, and chloromethyl ketone demonstrate impressive MMP inhibition. By inhibiting MMP activity, collagen and elastin may be preserved and wrinkles may be prevented.

### EXAMPLES

### Example 1

Human Gingival Fibroblasts (HGF-1) were purchased from ATCC (Manassas, Va.) and were grown in DMEM with Glutamax, 10% FBS and Penicillin/Streptomycin (All from Gibco) and kept at 37° C. in a humidified air chamber with 5% CO₂. Cells were seeded at 3×10⁵ cells/flask for the experiments and then grown to confluency. Cells between 3 and 10 passages were used for all experiments.

1,2,3,4,6-Penta-O-galloyl-β-D-glucose (purchased from Sigma-Aldrich) was dissolved in dimethylsulfoxide (DMSO) to obtain a 100 mM stock solution. Cells were simultaneously induced with PGG 10 μM, 50 μM or 100 μM, LPS 5 μg/mL or a combination, and the cells were allowed to continue growing in the incubator for 48 hours.

During the protein extraction, media was collected, flash frozen and saved for Western Blot and ELISA analysis. Cells were washed with cold PBS and lysed with RIPA buffer (150 mM NaCl, 50 mM Tris, 1% sodium deoxycholate, 1° A Triton X-100 and 0.1° A SDS). Cells were collected with a cell scraper and centrifuged at 14000 rpms for 30 minutes. The supernatant was collected and assayed for protein concentration. Samples were mixed with 4X SDS Loading buffer (40% glycerol, 8% SDS, 200 mM tris-HCl, 400 mM dithiothreitol, 0.005% bromophenol blue) for further analysis.

Gel electrophoresis was conducted using 12% sodium dodecyl sulfate (SDS) and polyacrylamide gels. The gels were prepared by standard methods and electrophoresed for 1 hour at 150 V.

Proteins from gels were transferred into 0.45 μm nitrocellulose paper (Bio-Rad) using tris-glycine transfer buffer with 20% methanol using 400 amp for 90 minutes. After transfer, membranes were removed from transfer boxes and rinsed in ddH₂O, and left to dry for 30 minutes. The nitrocellulose paper was blocked with Odyssey Blocking Buffer (PBS) (LI-COR Biosciences) in a rocker at room temperature for 1 hour.

After blocking, 0.1% Tween-20 was added, along with the primary antibody. Primary antibodies were diluted in blocking solution containing 0.1 Tween and incubated overnight at 4° C. with monoclonal antibody to MMP-1, MMP-3 (mouse, 1:2000 dilution; MAB901-MAB513, R&D Systems, Minneapolis, Minn.), MMP-2, MMP-9 (Cell Signaling Technology, Danvers, Mass.), MMP-8 and MMP-13 (Abcam, Cambridge, Mass.), and MMP-14 (EMD Millipore, Bilerica, Mass.). Blots were normalized by probing the membranes with Histone H3 (Cell Signaling Technology, Beverly, Mass.).

Incubation was done according to specific instructions from the manufacturer for each antibody. After incubation, membranes were washed three times with TBS-Tween. For the secondary antibody incubation, the membranes were placed in dark boxes with a mix of 1:1 blocking buffer and PBS containing 0.1% Tween-20 and the appropriate secondary antibody (LI-COR Biosciences) for 60 minutes, at room temperature. After incubation and washing with PBS-T, the membranes were incubated in the dark in blocking solution with 0.2% Tween, IRDye 800CW Goat anti-Rabbit IgG, and IRDye® 680RD Donkey anti-Mouse IgG (1:10000, LI-COR Biosciences, Lincoln, Nebr.) for 1 hour at room temperature. The proteins were detected and visualized by fluorescence using the Licor Odyssey Classic Infrared Imaging system (LI-COR Biosciences, Lincoln, Nebr.). Densitometry analysis of specific bands was performed with the Image Studio software provided by LI-COR Biosciences. Statistical analysis, including 1-way ANOVA with Dunnett's Multiple Comparison Test, was done using GraphPad Prism (GraphPad Software, La Jolla, Calif.).

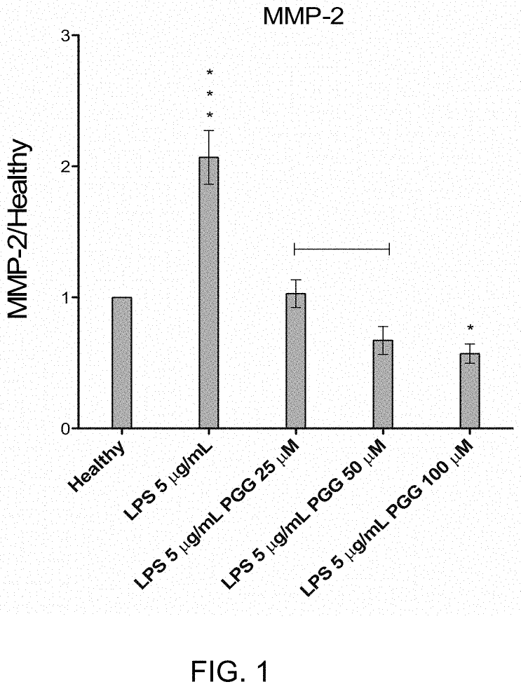

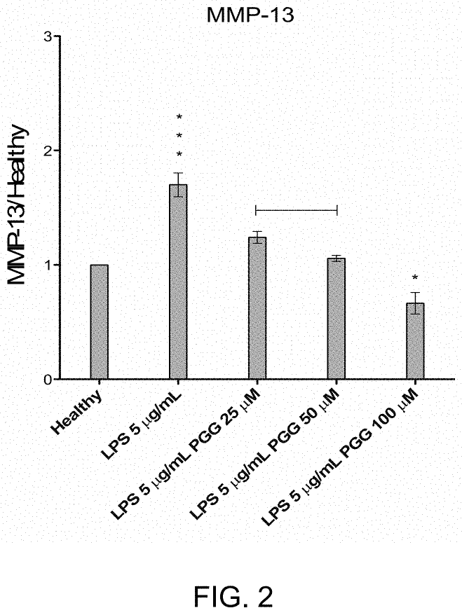

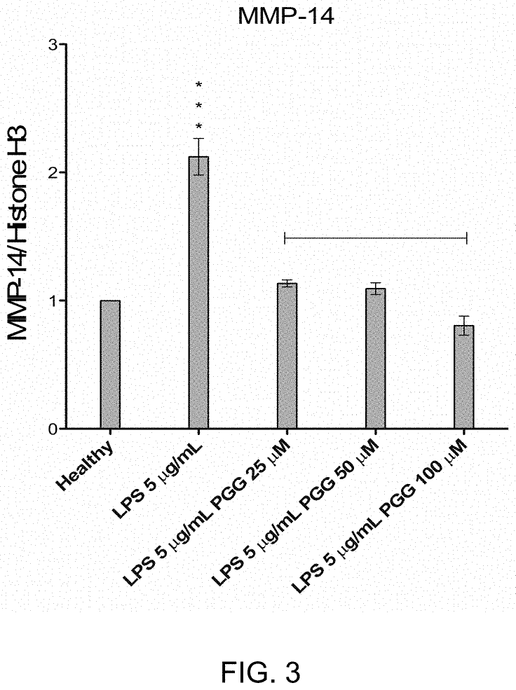

Human gingival fibroblasts (HGF-1) were treated with lipopolysaccharide (LPS) from P. gingivalis to reproduce the type of inflammatory response seen in chronic periodontitis. Upon treatment with PGG at different doses (25, 50 and 100 μM), MMP-2 was released into the pericellular space to be activated by MMP-14. Subsequently, MMP-2 and MMP-14 activate MMP-13. When induced with LPS alone, HGF-1 cells dramatically increased the amount of MMP-2 produced (FIG. 1). LPS activation of MMP-2, MMP-13 and MMP-14, however, was attenuated by the administration of PGG. At the lowest dose tested (25 μM), PGG demonstrated a reduction of these three MMPs to basal levels of production after LPS treatment. In cells treated with PGG 100 μM, levels of MMP-2 reduced significantly (p<0.01) compared to the healthy control. MMP-13 and MMP-14 (FIG. 2 and FIG. 3) expression in the treated groups was not significantly different than the healthy controls.

FIG. 1 shows that MMP-2 expression was induced by LPS (n=4 p<0.001, ANOVA, Dunnett's test). When simultaneously treated with LPS and increasing PGG concentrations, MMP-2 expression was not significantly different than in healthy controls. When treated with 100 μM PGG, MMP-2 levels were lower than in healthy controls (n=4 p<0.05, ANOVA, Dunnett's test). FIG. 2 shows a graphical representation of densitometry scans from four MMP-13 western blots in PGG-treated HGF-1 cells. After LPS induction, MMP-13 was upregulated (n=4, p<0.001, ANOVA, Dunnett's test). PGG treatment prevented upregulation of MMP-13 in a dose-dependent manner. FIG. 3 shows results from a Western blot analysis of MMP-14. Expression of MMP-14 increased significantly after 48 hour incubation with 5 μg/mL LPS (n=4, p<0.001, ANOVA, Dunnett's test). When the cells were treated with PGG and LPS together, MMP-14 levels were not significantly different from healthy controls. Error bars represent standard error.

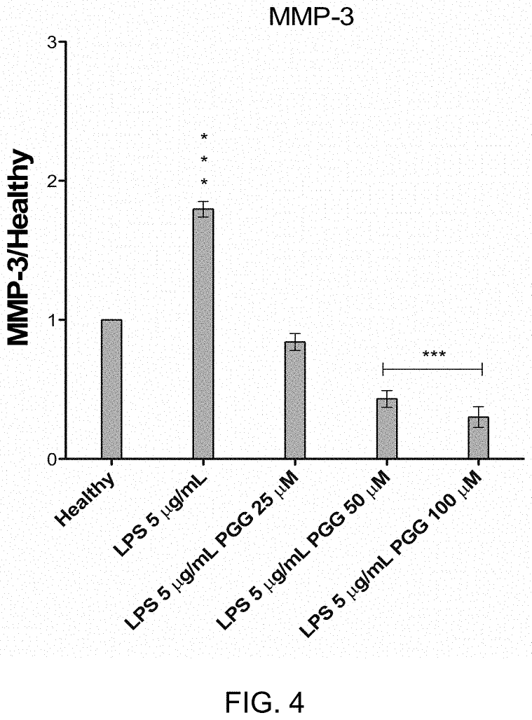

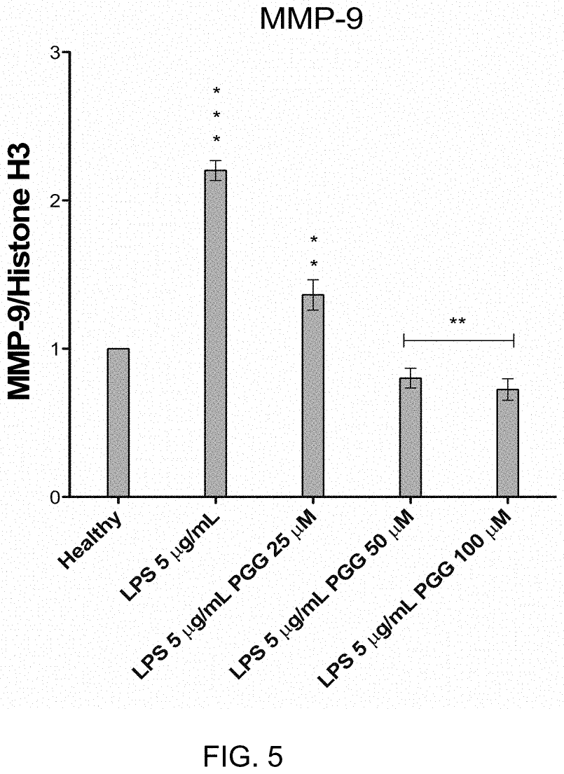

HGF-1 cells were treated with lipopolysaccharide (LPS, 5 μg/mL) from P. gingivalis in order to reproduce the type of inflammatory response seen in chronic periodontitis. Upon treatment with PGG at 25 μM, MMP-3 was comparable to healthy control values (FIG. 4). Increasing doses of 50 and 100 μM further reduced MMP-3 to levels below basal production (p<0.001). MMP-9, on the other hand, did not return to normal levels until it was treated with at least 50 μM PGG (FIG. 5), although a lower dose decreased the MMP-9 level when compared to the LPS inflammatory control.

### Example 2

Gene expression of MMPs was evaluated after treatment under different conditions. The tissue culture was washed with PBS. Cells were lysed and the RNA was then purified using an RNeasy mini kit (Cat# 74104) from Qiagen. All samples were treated with Qiagen DNase (Cat# 79254). One microgram of RNA was used for reverse transcription and subsequent SYBR® Green real time PCR for the genes of interest. Reverse transcription kits (Cat #330401) and SYBR Green real-time PCR master mixes (Cat# 330523) were from Qiagen (Louisville, Ky.).

The following primers and probes were used: Human MMP-2; MMP2 (Cat# PPH00151B), Human MMP-8; MMP8 (Cat# PPH00908C), Human MMP-13; MMP13 (Cat# PPH00121B), Human MMP-14; MMP14 (Cat# PPH00198C), and Human glyceraldehyde 3-phosphate dehydrogenase; GAPDH (Cat# PPH00150F).

Real time quantitative PCR was performed on an Applied Biosciences StepOne plus instrument and analyzed with StepOne software v2.3. The relative amounts of transcripts from each gene were normalized to reference gene GAPDH and calculated as follows: ΔΔC_{T}=the average ΔC_{T} of sample B−the average ΔC_{T} of sample B, and their fold difference=2^{-ΔΔ}C_{T} as previously described.

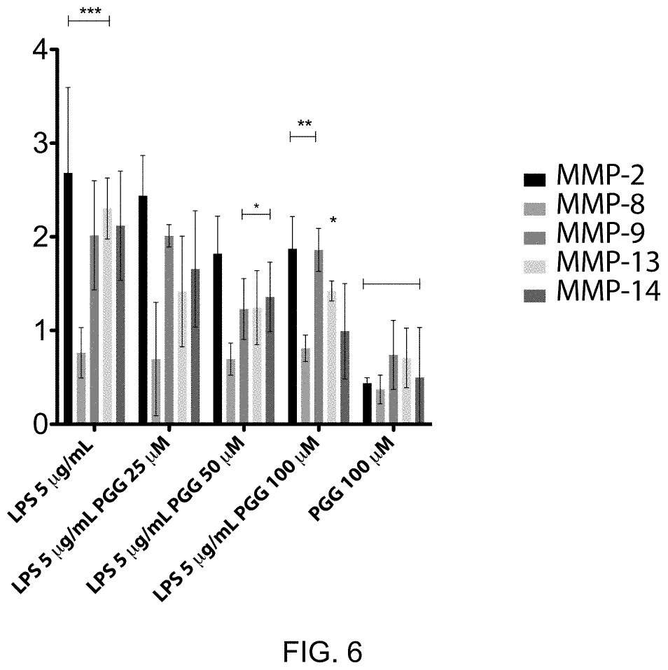

Our data suggests that treatment with PGG at 50-100 μM reduced MMP-2, MMP-9, MMP-13, and MMP-14 expression when compared with LPS induction with or without PGG 25 μM (FIG. 6). Treatment of healthy HGF-1 with PGG 100 μM did not significantly change MPP gene expression.

FIG. 6 shows increased expression of MMP-2, MMP-9 MMP-13 and MMP-14 genes after lypopolysaccharide (LPS) induction (5 μg/mL) (p<0.001, 2-way ANOVA, Bonferroni) (n=3). MMP-8 expression did not change significantly with LPS (5 μg/mL). Treatment with PGG 25 μM did not vary gene expression in a significant manner. Treatment with PGG 100 μM decreased the expression of MMP-2 compared to treatment with PGG 25-50 μM, yet expression was still higher compared with healthy HGF-1 (p<0.01, 2-way ANOVA, Bonferroni) (n=3). Treatment with PGG 100 μM significantly reduced the expression of MMP-9, MMP-13 and MMP-14 compared with induction with LPS alone (p<0.05, 2-way ANOVA, Bonferroni) (n=3).

### Example 3

MMP inhibition was tested using CMK and deferoxamine (DES). Ferric ammonium citrate solid (FAC, Amresco) was prepared in 1 mM hydrochloric acid to form a 50 mM stock solution. Iron solutions were freshly made for each experiment and the iron content quantified through a colorimetric assay. Cells were induced with FAC concentrations of 20, 50, and 100 μM, LPS and CMK or a combination, and the cells were allowed to grow in the incubator for 48 hours.

During the protein extraction, media was collected, flash frozen and saved for later analysis, including Western Blot and ELISA. Cells were washed with cold PBS and lysed with RIPA buffer (150 mM NaCl, 50 mM Tris, 1% Sodium deoxycholate, 1% Triton X-100 and 0.1% SDS). Cells were collected with a cell scraper and centrifuged at 14000 rpms for 30 minutes. The supernatant was collected and assayed for protein concentration. Samples were mixed with 4×SDS loading buffer (40% Glycerol, 8% SDS, 200 mM Tris-HCl, 400 mM dithiothreitol, 0.005% bromophenol blue) for further analysis.

Gel electrophoresis was conducted using 12% sodium dodecyl sulfate (SDS) and polyacrylamide gels. Equal amounts (30-50 μg) of protein were loaded in each lane and allowed to separate for 1 hour at 150 V.

Proteins from gels were transferred into 0.45 μm nitrocellulose paper (Bio-Rad) using tris-glycine transfer buffer with 20% methanol using 400 mAmp for 90 minutes. After transfer, membranes were removed from transfer boxes and rinsed in ddH2O, then left to dry for 30 minutes. The nitrocellulose paper was blocked with Odyssey Blocking Buffer (PBS) (LI-COR Biosciences) in a rocker at room temperature for 1 hour.

After blocking, 0.1% Tween-20 was added, along with primary antibody. Primary antibodies were diluted in blocking solution containing 0.1 Tween and incubated overnight at 4° C. with monoclonal antibody to MMP-1 (mouse, 1:2000 dilution; MAB901-MAB513, R&D Systems, Minneapolis, Minn.), MMP-2, MMP-9, MMP-8 and MMP-13 (Abcam, Cambridge, Mass.), and MMP-14 (EMD Millipore, Bilerica, Mass.). Blots were normalized by probing the membranes with Histone H3 as a control (Cell Signaling Technology, Beverly, Mass.).

Incubation periods were performed according to manufacturer instructions for each antibody. After incubation membranes were washed three times with TBS-tween. For secondary antibody incubation, membranes were placed in dark boxes with a mix of 1:1 blocking buffer and PBS containing 0.1% Tween-20 and the appropriate secondary antibody (LI-COR Biosciences) for 60 minutes, at room temperature. Once the incubation was finalized, blots were washed twice with TBS-tween and once with TBS. Membranes were scanned in a LI-COR Odyssey workstation for densitometric analysis.

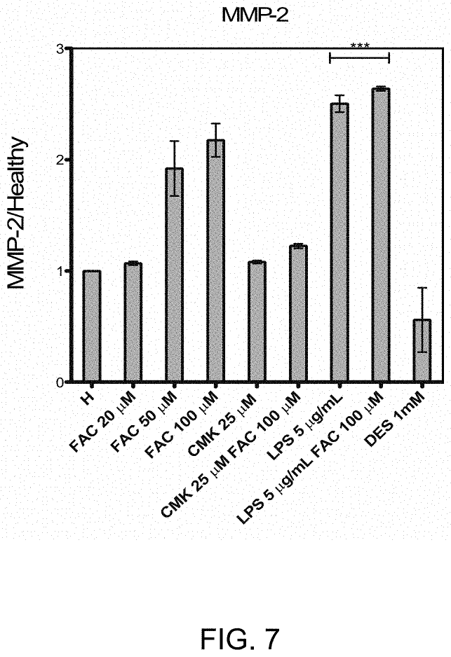

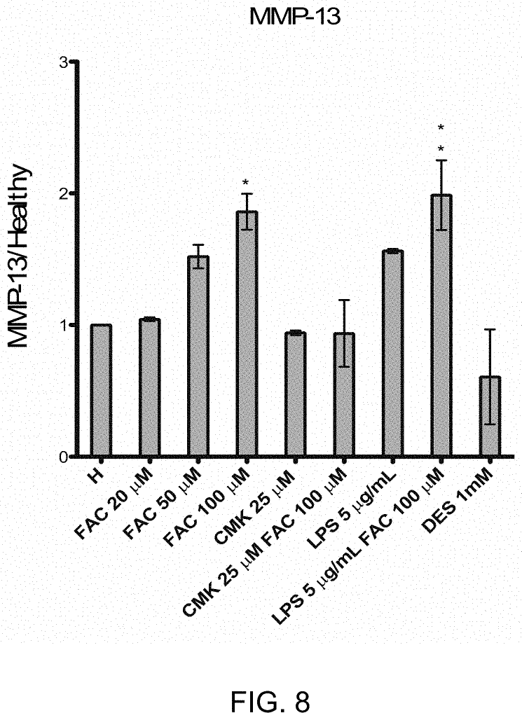

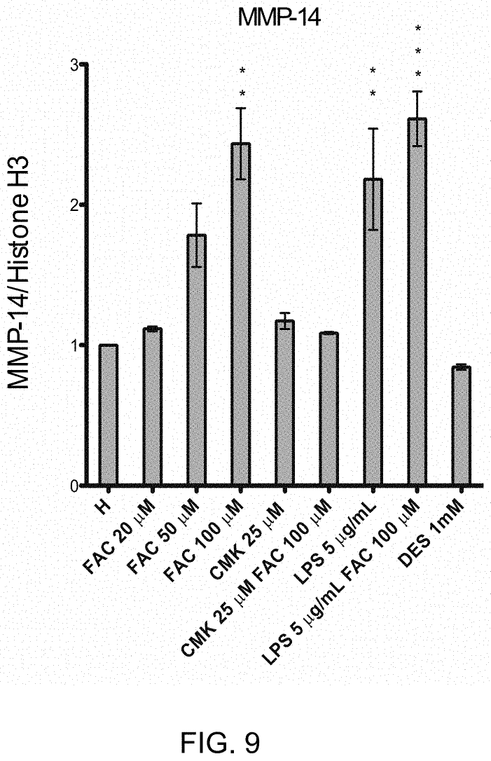

HGF-1 cells were incubated for 48 hours at differing concentrations of FAC (20, 50 and 100 μM). To evaluate the effect of FAC stimulation in the presence of inflammation, a control with LPS from P. gingivalis (5 μg/mL) was included, as well as a combination of LPS and FAC. We also included an additional control with chloromethylketone (CMK), a known inhibitor of PCSK3 the protease responsible for activation of proMMP-14 into MMP-14. Stimulation of the cells at the concentrations described previously resulted in a dose-dependent increased presence of MMP-2, MMP-13 and MMP-14 (FIG. 7, FIG. 8, and FIG. 9).

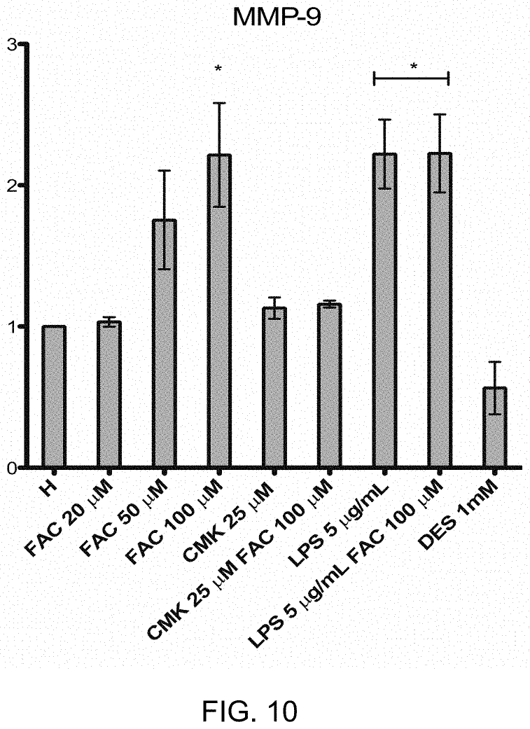

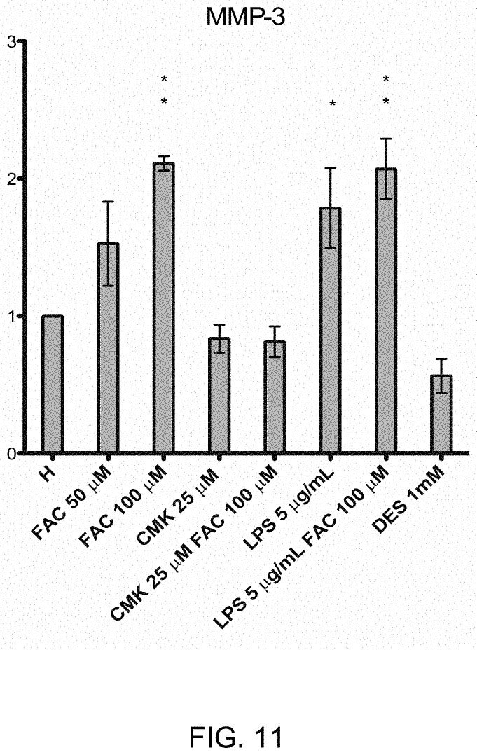

MMP-3 and MMP-9 are not subject to activation through PCSK3, yet show increased expression when HGF-1 cells are stimulated with FAC in the aforementioned conditions. (FIG. 10 and FIG. 11).

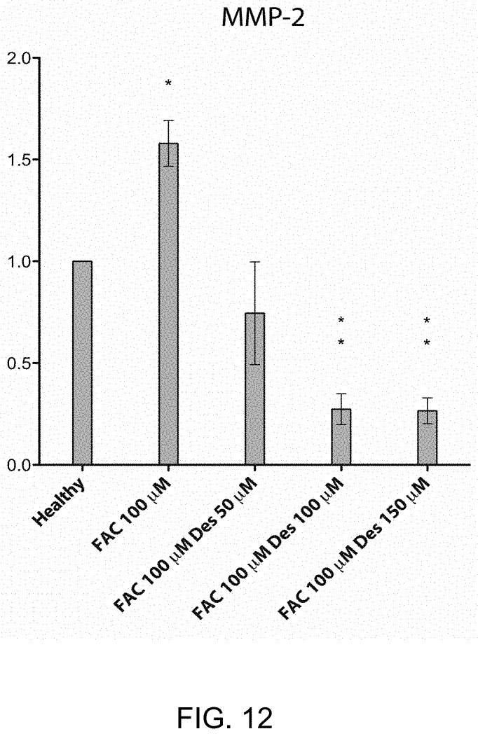

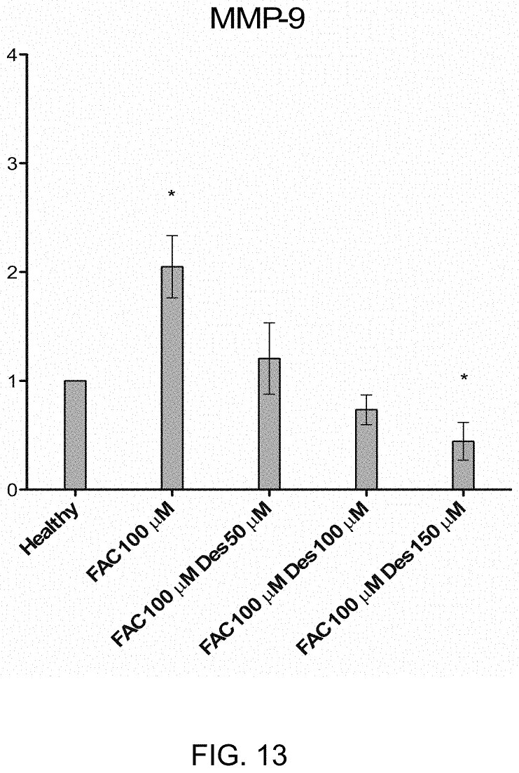

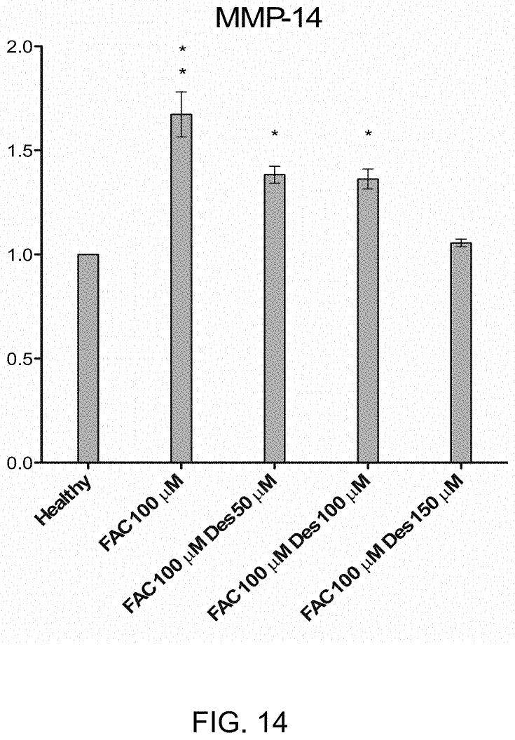

To better characterize the effectiveness of an iron chelator in regulating the upregulation of MMPs, HGF-1 cells were incubated with varying therapeutically relevant concentrations of deferoxamine (50, 100, 150 μM). Cell viability was unaffected at these concentrations, yet MMP secretion significantly decreased in a dose-dependent manner (FIG. 12, FIG. 13, and FIG. 14).

### Example 4

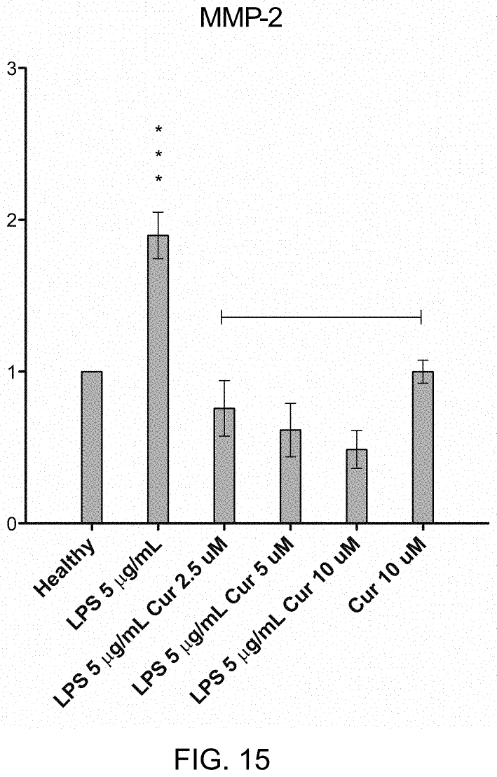

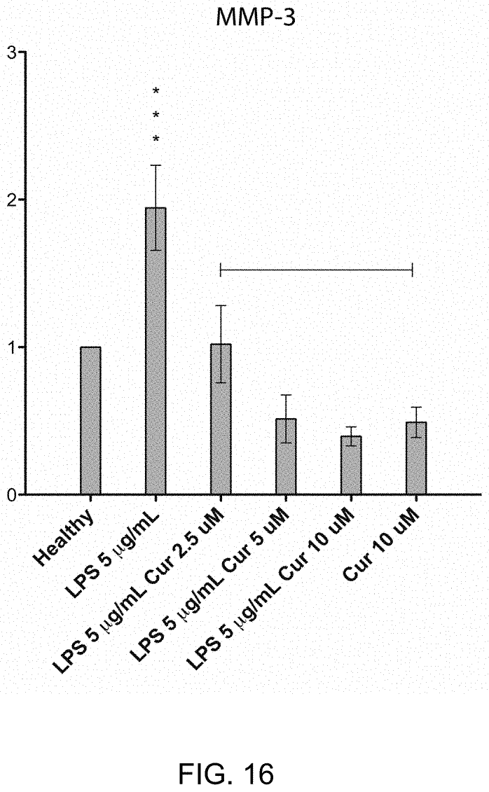

Curcumin decreased MMP-2 production in LPS-induced HGF-1 cells (FIG. 15). Curcumin also decreased MMP-3 production in LPS-induced HGF-1 cells (FIG. 16). HGF-1 cells were induced with LPS (5 μg/mL) to mimic the inflammatory process and Curcumin was added at different concentrations (2.5, 5 and 10 μM). LPS induction significantly increased MMP-2 (p<0.001, n=3) (1-way ANOVA, Bonferroni). Levels of MMP-2 after treatment with curcumin 2.5, 5, or 10 μM were not significantly different that the untreated HGF-1 (p_{>}0.05, n=3) (1-way ANOVA, Bonferroni). MMP-2 expression in HGF-1 treated with Curcumin 10 μM alone was not significantly different from untreated HGF-1 (p>0.05, n=3) (1-way ANOVA, Bonferroni).

### Example 5

Gene expression of MMPs was evaluated after treatment with CMK and deferoxamine. Tissue culture was washed with 1× PBS prior to collection of samples. Cells were lysed and RNA purified using an RNeasy mini kit (Cat# 74104) from Qiagen. RNA prep Kit was used according to manufacturer's protocol. All samples were treated with Qiagen DNase (Cat# 79254). One microgram of RNA was used for reverse transcription and subsequent SYBR® Green real time PCR for the genes of interest as previously described. Reverse transcription kits (Cat #330401) and SYBR Green real-time PCR master mixes (Cat# 330523) were purchased from Qiagen (Louisville, Ky.).

The following primers and probes were used: Human MMP-2; MMP2 (Cat# PPH00151B), Human MMP-8; MMP8 (Cat# PPH00908C), Human MMP-13; MMP13 (Cat# PPH00121B), Human MMP-14; MMP14 (Cat# PPH00198C), and Human glyceraldehyde 3-phosphate dehydrogenase; GAPDH (Cat# PPH00150F).

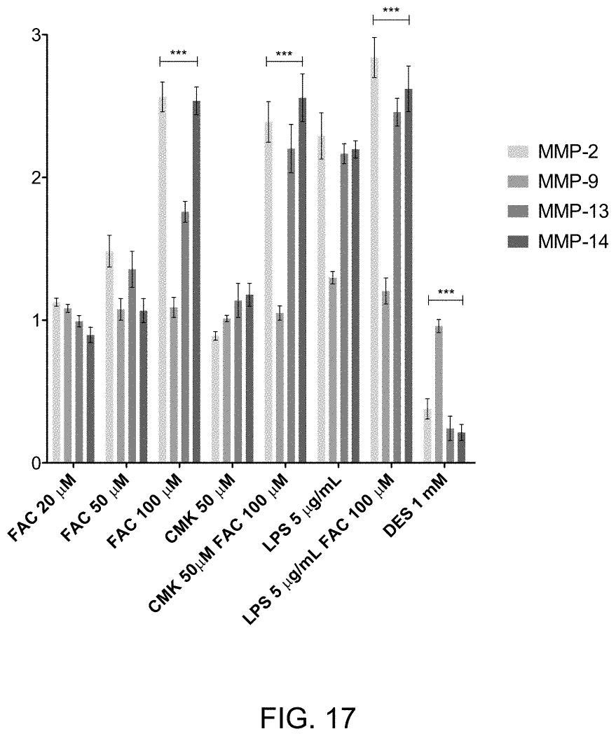

FIG. 17 is a summary of the qPCR analysis for each MMP. This is important because it showed the stimulation of mRNA by FAC. In contrast, the western blot analysis described above showed the actual production of the activated protein. Differences in the qPCR results and the western blot analysis indicated that the mRNA was made and potentially the precursor proMMP protein but if the proMMP was not activated by either PCSK3 or MMP-14, the protein might have been degraded.

The qPCR shows that 100 μM FAC acted as an inducer of gene expression of the MMPs in HGF-1 cells except for MMP-8. The qPCR in HGF-1 cells treated with 100 μM FAC showed expression of the MMP-2, MMP-3 and MMP-14 genes to make mRNA but the western blot analysis showed that treatment with CMK prevented an increase over healthy levels of the formation of the active forms of these MMPs. This suggests that inhibition of PCSK3 prevented the production of active MMPs even when the genes are activated and transcribed.

All of the compositions, materials, and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a device” is intended to include “at least one device” or “one or more devices.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is, therefore, intended that such changes and modifications be covered by the appended claims.

Statements

Statement 1: A topical, pharmaceutical composition, comprising a pharmaceutically acceptable carrier and a compound selected from the group consisting of 1,2,3,4,6-penta-O-galloyl-β-D-glucose, curcumin, deferoxamine, chloromethyl ketone, and any combination thereof.

Statement 2: The composition of statement 1, wherein the compound is 1,2,3,4,6-penta-O-galloyl-β-D-glucose.

Statement 3: The composition of statement 1, wherein the compound is curcumin.

Statement 4: The composition of statement 1, wherein the compound is deferoxamine.

Statement 5: The composition of statement 1, wherein the composition comprises a mixture of two or more of 1,2,3,4,6-penta-β-galloyl-β-D-glucose, deferoxamine, chloromethyl ketone, and curcumin.

Statement 6: The composition of any one of statements 1-5, wherein the compound or combination of compounds comprises from 0.00001% to 30% by volume of the composition.

Statement 7: The composition of any one of statements 1-6, wherein the pharmaceutically acceptable carrier is selected from the group consisting of poly(glycolide-co-dl-lactide) (PGLA), polyethylene glycol, collagen, hyaluronic acid, liposome, micelle, dendrimer, and any combination thereof.

Statement 8:The composition of any one of statements 1-7, wherein the pharmaceutically acceptable carrier is PGLA.

Statement 9: The composition of any one of statements 1-8, further comprising an antibiotic, anti-inflammatory, analgesic agent, and any combination thereof.

Statement 10: The composition of any one of statements 1-9, further comprising an antibiotic selected from the group consisting of tetracycline, doxycycline, metronidazole, clindamycin, amoxicillin and clavulinic acid, azithromycin, metronidazole, spiramycin, minocycline, and any combination thereof.

Statement 11: A method of treating periodontal disease, comprising administering to a subject the composition of any one of statements 1-10.

Statement 12: The method of statement 11, wherein the composition is administered orally.

Statement 13: The method of any one of statements 11-12, wherein the composition is administered sub-gingivally.

Statement 14: A method of treating a wound, fistula, or ulcer, comprising administering to a subject a topical composition of any one of statements 1-10.

Statement 15: The method of any one of statements 11-14, wherein the subject is a human.

Statement 16: The method of any one of statements 11-15, further comprising contacting keratinized, parakaratinized, or non-keratinized epithelium with the topical composition.

Statement 17: The method of any one of statements 11-16, wherein the periodontal disease is selected from the group consisting of gingivitis, periodontitis, and advanced periodontitis.

Statement 18: An ointment comprising the composition of any one of statements 1-10.

Statement 19: A mouthwash comprising the composition of any one of statements 1-10.

Statement 20: A syringe containing the composition of any one of statements 1-10.

Statement 21: A patch comprising the composition of any one of statements 1-10.

Statement 22: A method of preventing or treating wrinkles, comprising applying the composition of any one of statements 1-10 to skin.

What is claimed is:

1. A topical, pharmaceutical composition, comprising a pharmaceutically acceptable carrier and a compound selected from the group consisting of 1,2,3,4,6-penta-O-galloyl-β-D-glucose, curcum in, deferoxamine, chloromethyl ketone, and any combination thereof.

2. The composition of claim 1, wherein the compound is 1,2,3,4,6-penta-O-galloyl-β-D-glucose.

3. The composition of claim 1, wherein the compound is curcumin.

4. The composition of claim 1, wherein the compound is deferoxamine.

5. The composition of claim 1, wherein the composition comprises a mixture of two or more of 1,2,3,4,6-penta-O-galloyl-β-D-glucose, deferoxamine, chloromethyl ketone, and curcumin.

6. The composition of claim 1, wherein the compound or combination of compounds comprises from 0.00001% to 30% by volume of the composition.

7. The composition of claim 1, wherein the pharmaceutically acceptable carrier is selected from the group consisting of poly(glycolide-co-dl-lactide) (PGLA), polyethylene glycol, collagen, hyaluronic acid, liposome, micelle, dendrimer, and any combination thereof.

8. The composition of claim 1, wherein the pharmaceutically acceptable carrier is PGLA.

9. The composition of claim 1, further comprising an antibiotic, anti-inflammatory, analgesic agent, and any combination thereof.

10. The composition of claim 1, further comprising an antibiotic selected from the group consisting of tetracycline, doxycycline, metronidazole, clindamycin, amoxicillin and clavulinic acid, azithromycin, metronidazole, spiramycin, minocycline, and any combination thereof.

11. A method of treating periodontal disease, comprising administering to a subject the composition of claim 1.

12. The method of claim 11, wherein the composition is administered orally.

13. The method of claim 11, wherein the composition is administered sub-gingivally.

14. A method of treating a wound, fistula, or ulcer, comprising administering to a subject the topical pharmaceutical composition of claim 1.

15. The method of claim 14, wherein the subject is a human.

16. The method of claim 14, further comprising contacting keratinized, parakaratinized, or non-keratinized epithelium with the topical composition.

17. The method of claim 11, wherein the periodontal disease is selected from the group consisting of gingivitis, periodontitis, and advanced periodontitis.

18. An ointment comprising the composition of claim 1.

19. A mouthwash comprising the composition of claim 1.

20. A syringe containing the composition of claim 1.

21. A patch comprising the composition of claim 1.

22. A method of preventing or treating wrinkles, comprising applying the composition of claim 1 to skin.
